(19) United States
(12) Patent Application Publication
(10) Pub. No.: US 2023/0106315 A1
(43) Pub. Date: Apr. 6, 2023

(54) COMPOSITION COMPRISING TEXTURED LEGUMINOUS PROTEINS, METHOD FOR PREPARING SAME AND USE THEREOF

(71) Applicant: ROQUETTE FRERES, Lestrem (FR)

(72) Inventors: Charlotte DLUBAK, LESTREM (FR); Thomas BUCHE, LESTREM (FR); Cyril DROULEZ, LESTREM (FR); Florian SARRAZIN, LESTREM (FR)

(21) Appl. No.: 17/904,844

(22) PCT Filed: Feb. 26, 2021
(86) PCT No.: PCT/FR2021/050339

§ 371 Date: Aug. 23, 2022

(30) Foreign Application Priority Data
Feb. 28, 2020 (FR) 20 02039
Apr. 7, 2020 (FR) 20 03484

(51) Int. Cl. A23J 3/26; A23J 3/22; A23J 3/14; A23L 33/185; A23L 23/00

(52) U.S. Cl. CPC A23J 3/26; A23J 3/227; A23J 3/14; A23L 33/185; A23L 23/00

(57) ABSTRACT

The invention relates to a composition comprising leguminous proteins textured in a dry process, to a method for producing the same and to the use thereof.

The present invention relates to a specific composition comprising textured pea proteins, and to a method for the production thereof and to the use thereof.

The technique for texturing proteins, especially by extrusion cooking, with the aim of preparing products with a fibrous structure intended for producing meat and fish analogs, has been applied to numerous plant sources.

The extrusion cooking processes for proteins can be separated into two large families by the amount of water used in the process. When this amount is greater than 30% by weight, this will be referred to as “wet” extrusion cooking, and the products obtained will be more intended for producing finished products for immediate consumption that simulate animal meat, for example, beef steaks or chicken nuggets. For example, patent application WO 2014/081285 is known, which discloses a method for extruding a mixture of protein and fibers using a cooling die typical of wet extrusion. The present invention is in the field of dry extrusion.

When this amount of water is less than 30% by weight, this is then referred to as “dry” extrusion cooking: the products obtained are more intended to be used by food-processing manufacturers, in order to formulate meat substitutes by mixing them with other ingredients. The field of the present invention is that of “dry” extrusion cooking.

Historically, the first proteins used as meat analogs were extracted from soybean and wheat. Soybean subsequently quickly became the main source for this field of applications.

Patent application WO 2009/018548 is known, for example, which teaches that various mixtures containing proteins can be extruded in order to generate an extruded protein with aligned fibers allowing the simulation of meat fibers to be contemplated. However, no indication is provided concerning the influence of particle size, density or holding capacity on the application performance capabilities, or on the method used to produce them. Patent application US 2007/269567 specifies the particle sizes that are obtained (11 mm and 16.3 mm on average according to Table IV of Example 3).

While most of the studies that followed obviously related to soybean proteins, other sources of protein, both animal and plant, have been textured: peanut, sesame, cottonseed, sunflower, corn, wheat proteins, proteins derived from microorganisms, by-products from abattoirs or the fisheries industry.

Leguminous proteins, such as those derived from pea and faba bean, have also been the subject of work, both in terms of the isolation thereof and in terms of the “dry” extrusion cooking thereof.

Numerous studies have been undertaken on pea proteins given their particular functional and nutritional properties but also because of their non-genetically-modified nature.

Despite significant research efforts and increasing growth over recent years, the penetration of these products based on textured proteins on the food market is still subject to optimization.

One of the reasons particularly lies in the necessary process for rehydrating textured pea proteins before formulating them.

Indeed, since said proteins are dry, they must be rehydrated in order to be able to shape them and intimately mix them with other constituents of the formulation in order to obtain a satisfactory end result.

To this end, pea proteins textured in a dry process will be brought into contact with an aqueous solution. Unfortunately, the amount of water absorbed for the purposes of rehydration is not effective enough and, without additional human intervention, it is only approximately 50% of the amount required for the following formulation steps.

An additional step, called “shredding” or “cuttering” step, is therefore commonly carried out, which involves chopping up rehydrated textured fibers. The fibers obtained in this way are brought back into contact with an aqueous solution and, due to the chopping, will be able to reabsorb the required missing amount of water.

This step is complicated, since poorly managed chopping can damage the textured pea proteins. In addition, it is an additional preparation step, which makes implementation more complex.

One solution involves reducing the size of the particles of textured proteins, from the production stage. This size reduction optimizes the water uptake of textured proteins due to the increased protein/water exchange surface. The shredding step after rehydration becomes unnecessary, due to the reduction in particle size achieved as soon as the textured protein is produced.

Unfortunately, the reduction in the particle size of the textured proteins affects the organoleptic properties of the final meat or fish analogs, made with said textured vegetable proteins. The article entitled “Effect of soy particle size and color on the sensory properties of ground beef patties” (Cardello & al., Journal of food quality, 1983) presents the organoleptic consequences in FIG. 3. This study aimed to study the organoleptic impact of various sizes of textured soybean proteins in beef. It can be seen that the best results are obtained, without achieving the results of beef, with textured soybean proteins with a particle size of more than 9.52 mm that represents more than 73% of the total particles. Any reduction in this particle size distribution will involve a reduction in the reproduction of the organoleptic qualities of the meat analog that is obtained.

This decrease in the organoleptic result can be explained by the disappearance of the amount and the integrity of the matter required to emulate the fibers of meats. As the particles are smaller, the fibers obtained in the meat or fish analog no longer have sufficient effective fiber sizes.

In order to overcome this problem, a potential solution involves increasing the density of textured plant proteins in order to overcome the small size of protein fibers, by densifying them. Short but denser protein fibers would thus have a firmer structure, better simulating the organoleptic result to be achieved.

This strategy unfortunately has a significant impact on the water holding capacity of a textured vegetable protein. The article entitled “EXTRUSION OF TEXTURIZED PROTEINS” (Kearns & al., American Soybean Association) presents the direct link established between density and water holding capacity (WHC). It can be clearly seen that the water holding capacity drops as the density increases. A textured soybean protein with a density of 216 WI thus has a water holding capacity of just over 3 g of water per gram of protein, and always less than 3.5. Any increase in density causes this water holding capacity to drop, sometimes below 2.

This negative correlation between density and water holding capacity is also clearly demonstrated in Table 1 of the article entitled “Effect of Value-Enhanced Texturized Soy Protein on the Sensory and Cooking Properties of Beef Patties” (A. A. Heywood et al., JAOCS, Vol. 79, No. 7, 2002). These data therefore confirm that high density implies low water holding capacity and vice versa. Obtaining a textured protein with both high density and high water holding therefore seems impossible. However, such a product is of interest in the industry.

It is to the Applicant's credit that they have solved the above problems and have developed a new specific composition comprising textured leguminous proteins, obtained by extrusion cooking in a dry process, the particle size of which is reduced, the density is high and the water holding capacity is improved, while retaining a textured protein yielding excellent results in meat and fish analog applications.

This invention will be better understood in the following section which aims to disclose a general description thereof.

### GENERAL DESCRIPTION OF THE PRESENT INVENTION

The present invention relates to a composition comprising leguminous proteins textured in a dry process in the form of particles, the composition having a water holding capacity measured by a test A of more than 3.5 g of water per g of dry proteins, preferably ranging between 3.5 and 4.5 g of water per g of dry proteins, even more preferably ranging between 3.5 and 4 g of water per g of dry proteins, a density measured by a test B ranging between 190 and 230 WI and at least 85% of the textured leguminous protein particles being between 2 mm and 5 mm in size.

Preferably, the leguminous protein is selected from the list made up of faba bean and pea. Pea is particularly preferred.

The protein content within the composition ranges between 60% and 80%, preferably between 70% and 80% by dry weight relative to the total weight of dry matter of the composition.

Finally, the dry matter of the leguminous protein textured in a dry process according to the invention is more than 80% by weight, preferably more than 90% by weight.

The present invention also relates to a method for producing a composition of leguminous proteins as described above, characterized in that the method comprises the following steps:
1) providing a powder comprising leguminous proteins and leguminous fibers, having a dry weight ratio of leguminous proteins to leguminous fibers ranging between 70/30 and 90/10, preferably ranging between 75/25 and 85/15;
2) extrusion cooking the powder with water, the water to powder mass ratio before cooking ranging between 20% and 40%, preferably between 25% and 35%, even more preferably 30%;
3) cutting the extruded composition at the extruder outlet made up of an outlet die with holes, with a diameter of 1.5 mm and equipped with a knife, the speed of rotation of which ranges between 1,200 and 1,800 revolutions per minute, or between 2,000 and 2,400 revolutions per minute, preferably around 1,500 revolutions per minute;
4) drying the composition thus obtained.

Preferably, the leguminous protein used in the method according to the invention is selected from the list comprising faba beans and peas, preferably a pea protein.

The powder comprising the leguminous proteins and leguminous fibers used in step 1 can be prepared by mixing said proteins and fibers. The powder can essentially consist of leguminous proteins and leguminous fibers. The term “essentially consist of” means that the powder can comprise impurities associated with the method for producing the proteins and the fibers, for example, traces of starch. Preferably, the leguminous protein and fiber are selected from the list made up of faba bean and pea. Pea is particularly preferred.

Preferably, step 2 is carried out by extrusion cooking in a twin-screw extruder characterized by a length to diameter ratio ranging between 20 and 45, preferably between 35 and 45, preferably 40, and equipped with 85-95% feeding elements, 2.5-10% kneading elements, and 2.5-10% reverse pitch elements.

Even more preferably, a specific power ranging between 10 and 25 kWh/kg is applied to the powder mixture, by regulating the pressure at the outlet in a range ranging between 10 and 25 bars, preferably between 12 and 16 bars or between 17 and 23 bars.

Even more preferably, the output of the twin-screw extruder is made up of an output die with holes with a diameter of 1.5 mm and with a knife, the speed of rotation of which ranges between 1,200 and 1,800 revolutions per minute or between 2,000 and 2,400 revolutions per minute, preferably 1,500 revolutions per minute.

Finally, the present invention relates to the use of the composition of leguminous proteins textured in a dry process as described above in industrial applications such as, for example, the human and animal food industry, industrial pharmaceuticals or cosmetics.

Preferably, the leguminous protein used in these applications is a pea protein.

The present invention will be better understood upon reading the following detailed description.

### DETAILED DESCRIPTION OF THE PRESENT INVENTION

The present invention relates to a composition comprising leguminous proteins textured in a dry process in the form of particles, the composition having a water holding capacity measured by a test A of more than 3.5 g of water per g of dry proteins, preferably ranging between 3.5 and 4.5 g of water per g of dry proteins, even more preferably ranging between 3.5 and 4 g of water per g of dry proteins, a density measured by a test B ranging between 190 and 230 g/l and at least 85% of the textured leguminous protein particles being between 2 mm and 5 mm in size.

The leguminous protein is preferably selected from the list made up of faba bean protein and pea protein. Pea protein is particularly preferred.

The term “leguminous” is considered herein to mean the family of dicotyledonous plants of the order Fabales. This is one of the largest flowering plant families, third after Orchidaceae and Asteraceae in terms of number of species. It contains approximately 765 genera, bringing together more than 19,500 species. Several leguminous plants are important crop plants, including soybean, beans, peas, faba beans, chickpeas, peanuts, cultivated lentils, cultivated alfalfa, various clovers, broad beans, carob and licorice.

The term “pea” is considered here in its broadest accepted use and includes in particular all the varieties of “smooth pea” and “wrinkled pea” and all the mutant varieties of “smooth pea” and “wrinkled pea”, regardless of the uses for which said varieties are usually intended (human food, animal feed and/or other uses).

The term “pea” in the present application includes pea varieties belonging to the Pisum genus and more specifically to the sativum and aestivum species. Said mutant varieties are in particular those called “mutants r”, “mutants rb”, “mutants rug 3”, “mutants rug 4”, “mutants rug 5” and “mutants lam” as described in the article by C-L HEYDLEY et al., entitled “Developing novel pea starches”, Proceedings of the Symposium of the Industrial Biochemistry and Biotechnology Group of the Biochemical Society, 1996, pp. 77-87.

If the leguminous proteins, in particular derived from faba beans and peas, are particularly adapted to the design of the invention, it is nevertheless possible to achieve the latter with other sources of plant proteins such as oat, mung bean, potato, corn or even chickpea protein. A person skilled in the art will know how to make any necessary adjustments.

“Textured” or “texturing” in the present application is understood to mean any physical and/or chemical process that aims to modify a composition comprising proteins in order to give it a specific ordered structure. Within the scope of the invention, texturing proteins aims to give the appearance of a fiber, such as those present in animal meats. As will be described throughout the remainder of this description, a particularly preferred method for texturing proteins is extrusion cooking, particularly using a twin-screw extruder.

In order to measure the water holding capacity, test A is used, the protocol of which is described below:

a. weighing a 20 g sample to be analyzed in a beaker;
b. adding drinking water at room temperature (temperature between 10° C. and 20° C., preferably 20° C.+/−1° C.) until the sample is completely submerged;
c. leaving in static contact for 30 minutes;
d. leaving to drain;
e. separating the residual water and the sample using a sieve;
f. weighing the final weight P of the rehydrated sample.

The computation for water holding capacity, expressed as grams of water per gram of protein analyzed, is as follows:

Water holding capacity=(P−20)/20.

“Drinking water” is understood to mean water that can be drunk or used for domestic and industrial purposes without posing health risks. Preferably, its conductivity is selected between 400 and 1,100, preferably between 400 and 600 μS/cm. More preferably in the present invention, it will be understood that this drinking water has a sulfate content of less than 250 mg/l, a chloride content of less than 200 mg/l, a potassium content of less than 12 mg/l, a pH ranging between 6.5 and 9 and a total hardness (TH, namely the hardness of the water, corresponding to the measurement of the calcium and magnesium ions content in water) of more than 15 French degrees. In other words, drinking water must not have less than 60 mg/l of calcium or 36 mg/l of magnesium.

In order to measure the density, test B is used, the protocol of which is described below:
a. taring a 2 liter graduated test tube;
b. filling the test tube with the product to be analyzed, until the 2 liter graduation is reached;
c. weighing the product (Weight P, in grams).

The computation of the density expressed in g/l is as follows:

Density=(P(g)/2).

The protocol for determining the size of the constituent particles measured according to a test C, expressed as a percentage, is as follows: —A system of sieves stacked on a machine is used that allows said sieves to be stirred, in order to circulate the particles through the meshes. A particularly suitable commercial reference is the Electromagnetic laboratory sieve machine, the Analyette 3 model, marketed by FRITSCH.

The various sieves that are used are: 1 mm, 2 mm, 5 mm, 10 mm.
100 g of product is introduced at the top and the device is set to vibration mode for 3 min. This time can be changed, provided that particle size separation is complete.
After stopping, the weight of each fraction accumulated on each sieve is weighed, which is called the “refusal” of the sieve. It is in fact the particles that have failed to pass through the mesh as they are too big.
The computation is as follows:
   larger than 10 mm=(refusal weight 10 mm/weight X)*100;
   between 5 and 10 mm=(refusal weight 5 mm/Weight X)*100;
   between 2 and 5 mm=(refusal weight 2 mm/Weight X)*100;
   between 1 and 2 mm=(refusal weight 1 mm/Weight X)*100;
   smaller than 1 mm=(final refusal weight/Weight X)*100.

As indicated above, the textured pea protein compositions of the prior art are already well known and used in the food industry, in particular in meat analogs. In order to use them in a recipe, it is known that the required water content is at least 3 g per g of proteins, with 4 g being preferred. This rehydration will make it possible to prepare the fibers to be included in the formulation, by best simulating the functional properties of meat fibers, and will avoid the excessive presence of poorly rehydrated parts, causing a sensation of hardness, even of crunchiness during final consumption. It is also known that this rehydration cannot be carried out in a single step.

A person skilled in the art, aware of the problem of water uptake of textured proteins, will first carry out a first rehydration step by placing the textured pea protein with an aqueous solvent, reaching approximately 2 g of water per g of proteins. They will then shred the rehydrated protein fibers. Without wishing to be bound by a particular theory, this “shredding” will allow the fibers to be destructured and thus expose the internal parts and enable the hydration thereof. The rehydrated and destructured protein fibers in contact with the aqueous solvent will simply need to be replaced, the water holding capacity will be more than 3.5 g per g of proteins.

For example, the indication of this requirement for the shredding step is found in the NUTRALYS® T70S technical documentation produced and marketed by the applicant (refer to the extract “Recipe preparation includes a shredding step of NUTRALYS® T70S” cited in the following link: https://www.roquette.com/-/media/contenus-gbu/food/plant-proteins—concepts/roquette-food-breakfast-sausage-us-2020-04-1511-(1).pdf).

Shredding proteins is a well known solution, but it adds a step, making the final formulation process more complex, and causing an increase in costs. Moreover, if this shredding is poorly managed, it will cause excessive destructuring of the fibers, causing a loss of the desired functional effects. Since the plant fibers have been shortened, they will not simulate meat fibers as well.

Finally, the dry matter of the leguminous protein textured in a dry process according to the invention is more than 80% by weight, preferably more than 90% by weight.

The dry matter is measured using any method that is well known to a person skilled in the art. Preferably, the “desiccation” method is used. It involves determining the amount of water evaporated by heating a known amount of a sample of known mass. Heating is continued until the mass stabilizes, indicating that the water has evaporated completely. Preferably, the temperature used is 105° C.

The protein content of the composition according to the invention advantageously ranges between 60% and 80%, preferably between 70% and 80% by weight relative to the total dry matter. Any method well known to a person skilled in the art can be used to analyze this protein content. Preferably, the total nitrogen amount will be assayed and this content will be multiplied by the coefficient 6.25. This method is particularly known and used for plant proteins.

The present invention also relates to a method for producing a composition of leguminous proteins as described above, characterized in that the method comprises the following steps:

1) providing a powder comprising leguminous proteins and leguminous fibers, having a dry weight ratio of leguminous proteins to leguminous fibers ranging between 70/30 and 90/10, preferably ranging between 75/25 and 85/15;
2) extrusion cooking the powder with water, the water to powder mass ratio before cooking ranging between 20% and 40%, preferably between 25% and 35%, even more preferably 30%;
3) drying the composition thus obtained.

Preferably, the leguminous protein and the leguminous fiber of step 1 are selected from the list made up of faba bean protein and pea protein. Pea protein is particularly preferred.

The powder comprising the leguminous proteins and leguminous fibers used in step 1 can be prepared by mixing said proteins and fibers. The powder can essentially consist of leguminous proteins and leguminous fibers. The term “essentially consist of” means that the powder can comprise impurities associated with the method for producing the proteins and the fibers, for example, traces of starch. Mixing involves obtaining a dry mixture of the various constituents required to synthesize the plant fiber during step 2.

Preferably, the leguminous proteins are characterized by a protein content advantageously ranging between 60% and 90%, preferably between 70% and 85%, even more preferably between 75% and 85% by weight to the total dry matter. Any method well known to a person skilled in the art can be used to analyze this protein content. Preferably, the total nitrogen amount will be assayed and this content will be multiplied by the coefficient 6.25. This method is particularly known and used for plant proteins. Preferably, the dry matter of the leguminous protein is more than 80% by weight, preferably more than 90% by weight.

Even more preferably, the leguminous proteins are characterized by a solubility at pH 3 of more than 30%. The solubility is measured using the following protocol: a suspension of the powder at 2.5% w/w is produced with distilled water with an amount Q1, the pH is adjusted to the desired value, it is stirred for 30 minutes at 1,100 rpm using a magnetic bar, centrifugation is carried out for 15 minutes at 3,000 g and then the amount of material Q2 in the supernatant is analyzed using its weight and dry matter (obtained, for example, using the method known as “dessication”. It involves determining the amount of water evaporated by heating a known amount of a sample of known mass. The heating is continued until the mass stabilizes, indicating that the evaporation of the water is complete. Preferably, the temperature used is 105° C.). The solubility is obtained by the formula: (Q2/Q1)*100 d.

Even more preferably, the proteins are characterized by a particle size characterized by a Dmode ranging between 150 microns and 400 microns, preferably between 150 microns and 200 microns or between 350 microns and 450 microns. The measurement of this particle size is carried out using a MALVERN 3000 laser particle size analyzer in the dry phase (equipped with a powder module). The powder is placed in the feeder for the module with an opening ranging between 1 and 4 mm and a vibration frequency of 50% or 75%. The device automatically records the various sizes and adjusts the Particle Size Distribution (or PSD) as well as the Dmode, D10, D50 and D90. The Dmode is well known to a person skilled in the art and consists of the size of the largest population of particles.

The particle size of the powder is advantageous for the stability and the productivity of the method. An excessively fine particle size is irrevocably followed by problems that are sometimes difficult to manage during the extrusion method.

“Leguminous fiber” is understood to mean any compositions comprising polysaccharides that are relatively indigestible or indigestible by the human digestive system, extracted from leguminous plants. Such fibers are extracted using any method that is well known to a person skilled in the art.

Preferably, the leguminous fiber is derived from a pea using a wet extraction method. The dehulled pea is reduced to flour, which is then suspended in water. The suspension thus obtained is sent to hydrocyclones in order to extract the starch. The supernatant is sent to horizontal settling tanks in order to obtain a leguminous fiber fraction. Such a method is described in patent application EP 2950662. A leguminous fiber thus prepared contains between 40% and 60% of polymers made up of cellulose, hemicellulose and pectin, preferably between 45% and 55%, as well as between 25% and 45% of pea starch, preferably between 30% and 40%. A commercial example of such a fiber is, for example, the Pea Fiber 150 fiber by Roquette.

The mixing can be carried out upstream using a dry mixer or even directly as a feed from step 2. During this mixing, additives can be added that are well known to a person skilled in the art, such as flavorings or even dyes.

In an alternative embodiment, the fiber/protein mixture is naturally obtained by turboseparation of a leguminous flour. The leguminous plant seeds are cleaned, their outer fibers are removed, and they are ground to flour. The flour is then turboseparated, which consists in applying a rising stream of air, enabling the different particles to be separated based on their density. This thus makes it possible to concentrate the content of proteins in the flours from approximately 20% to more than 60%. Such flours are called “concentrates”. These concentrates also contain between 10% and 20% of leguminous fibers.

The dry weight ratio between proteins and fibers is advantageously between 70/30 and 90/10, preferably between 75/25 and 85/15.

During step 2, this mixture of powders will then be textured, which is the same as saying that the proteins and fibers will undergo thermal destructuring and reorganization in order to form fibers with continuous elongation in straight, parallel lines, simulating the fibers present in meats. Any method well known to a person skilled in the art will be suitable, in particular extrusion.

Extrusion consists in forcing a product to flow through a small hole, the die, under the action of high pressures and shearing forces, using the rotation of one or two Archimedes screws. The resulting heating causes cooking and/or denaturing of the product, hence the term sometimes used, “extrusion cooking”, then expansion by evaporation of the water at the die outlet. This technique makes it possible to develop products which are widely varied in their composition, their structure (expanded and alveolar form of the product), and their functional and nutritional properties (denaturing of anti-nutritional or toxic factors, sterilization of food, for example). Processing of proteins often leads to structural modifications which are reflected by obtaining products with a fibrous appearance, simulating animal meat fibers. Step 2 must be carried out with a water to powder mass ratio before cooking ranging between 20% and 40%, preferably between 25% and 35%, even more preferably 30%. This ratio is obtained by dividing the amount of water by the amount of powder, and by multiplying by 100. Preferably, the water is injected at the end of the feeding zone and immediately before the kneading zone.

Without being bound by any theory, it is well known to a person skilled in the art of extrusion cooking that it is this ratio that will allow the required density to be obtained. The values of this ratio therefore will potentially be 20, 21, 22, 23, 24, 25, 26, 27, 28, 29, 30, 31, 32, 33, 34, 35, 36, 37, 38, 39 or 40%.

Any drinking water is suitable for this purpose. “Drinking water” is understood to mean water that can be drunk or used for domestic and industrial purposes without posing health risks. Preferably, its conductivity is selected between 400 and 1,100, preferably between 400 and 600 μS/cm. More preferably in the present invention, it will be understood that this drinking water has a sulfate content of less than 250 mg/l, a chloride content of less than 200 mg/l, a potassium content of less than 12 mg/l, a pH ranging between 6.5 and 9 and a total hardness (TH, namely the hardness of the water, corresponding to the measurement of the calcium and magnesium ions content in water) of more than 15 French degrees. In other words, drinking water must not have less than 60 mg/l of calcium or 36 mg/l of magnesium. This definition includes water from the drinking water network, decarbonated water, demineralized water.

Preferably, step 2 is carried out by extrusion cooking in a twin-screw extruder characterized by a length to diameter ratio ranging between 20 and 45, preferably between 35 and 45, preferably 40, and equipped with a series of 85-95% feeding elements, 2.5-10% kneading elements, and 2.5-10% reverse pitch elements.

The length to diameter ratio is a conventional parameter in extrusion cooking. This ratio therefore can be 20, 21, 22, 23, 24, 25, 26, 27, 28, 29, 30, 31, 32, 33, 34, 35, 36, 37, 38, 39, 40, 41, 42, 43, 44 or 45.

The various elements are the feeding elements intended for feeding the product into the die without modifying the product, the kneading elements intended for mixing the product and the reverse pitch elements intended for applying a force to the product to cause it to advance in the opposite direction and thus cause mixing and shearing.

Preferably, the feeding elements will be placed at the very beginning of the screw with a temperature set between 20° C. and 70° C., then the kneading elements with a temperature ranging between 90° C. and 150° C. and finally the reverse pitch elements with temperatures ranging between 100° C. and 120° C.

Preferably, this screw is rotated between 900 and 1,200 revolutions/min, preferably between 1,000 and 1,100 revolutions/min.

Even more preferably, a specific power ranging between 10 and 25 kWh/kg is applied to the powder mixture, by regulating the pressure at the outlet in a range ranging between 10 and 25 bars, preferably between 12 and 16 bars or between 17 and 23 bars.

Step 3 then involves cutting the extruded composition at the extruder outlet made up of an outlet die with holes, with a diameter of 1.5 mm and equipped with a knife, the speed of rotation of which ranges between 1,200 and 1,800 revolutions per minute, or between 2,000 and 2,400 revolutions per minute, preferably around 1,500 revolutions/min.

The knife is placed flush with the outlet of the extruder, preferably at a distance ranging between 0 and 5 mm. “Flush” is understood to be at a distance extremely close to the die located at the outlet of the extruder, at the limit of touching the die but without touching it. Conventionally, a person skilled in the art will adjust this distance by making the knife and the die touch each other, then by shifting the latter very slightly.

The last step 4 involves drying the composition thus obtained.

A person skilled in the art will know how to use the appropriate technology in order to dry the composition according to the invention from the wide selection currently available to them. Without limitation and solely by way of an example, air flow dryers, microwave dryers, fluidized bed dryers or vacuum dryers can be cited. A person skilled in the art will select the correct parameters, mainly the time and temperature, in order to achieve the desired final dry matter.

Finally, the present invention relates to the use of the composition of leguminous proteins textured in a dry process as described above in industrial applications such as, for example, the human and animal food industry, industrial pharmaceuticals or cosmetics.

The human and animal food industry is understood to mean industrial confectionery (for example, chocolate, caramel, jelly sweets), bakery products (for example, bread, brioches, muffins), the meat and fish industry (for example, sausages, hamburgers, fish nuggets, chicken nuggets), sauces (for example, bolognaise, mayonnaise), products derived from milk (for example, cheese, plant milk), beverages (for example, high protein beverages, powdered beverages to be reconstituted).

More preferably, the present invention relates to the use of the composition of leguminous proteins textured in a dry process as described above in the field of baking.

The invention will be of particular interest in order to produce inclusions in bakery products such as muffins, cookies, cakes, bagels, pizza dough, breads and breakfast cereals.

The term “inclusions” is understood to mean particles (in this case the composition of leguminous proteins textured in a dry process) mixed with a dough before it is cooked. After this step, the composition of leguminous proteins textured in a dry process is trapped in the final product (hence the term “inclusion”) and provides both its protein content as well as crunchiness when consumed.

The invention will be of particular interest in order to produce inclusions in confectionery products such as fat filings, chocolates, so as to also provide protein retention as well as crunchiness.

The invention will be of particular interest in order to produce inclusions in products that are alternatives to dairy products such as cheeses, yogurts, ice creams and beverages.

The invention will be of particular interest in the field of analogs of meat, fish, sauces, soups.

A particular application relates to the use of the composition according to the invention for manufacturing meat substitutes, in particular minced meat. Yet also bolognaise sauce, steak for hamburgers, meat for tacos and pitta, “Chili sin came”.

In pizzas, the composition comprising textured leguminous proteins according to the invention will be of particular interest for being sprinkled on top of said pizza (“topping”).

In dehydrated ready meals (for example, Bolino in Europe or Good Dot in India), the textured composition according to the invention will be used as an element providing fiber and protein. Thus, a product can be obtained that hydrates quickly and to its core, while being pleasing to chew.

The invention will be better understood upon reading the following non-limiting examples.

### EXAMPLES

### Example 1: Production of a Composition of Leguminous Proteins Textured in a Dry Process According to the Invention

A powder mixture consisting of 87% of NUTRALYS® F85M pea protein (comprising 87.2% of proteins) by ROQUETTE and 12.5% of 150M pea fiber is produced. The protein content in 100 g of mixture is therefore 87*0.872=75.9 g.

This mixture is introduced by gravity into a COPERION ZSK 54 MV extruder from COPERION.

The mixture is introduced with a regulated flow rate of 300 kg/h. An amount of 78 kg/h of water is also introduced. The water to powder mass ratio is therefore (78/300)*100=26%.

The extrusion screw, made up of 85% feeding elements, 5% kneading elements and 10% reverse pitch elements, is rotated at a speed of 1,000 rpm and sends the mixture to a die. As indicated in the description, the feeding elements were placed at the very beginning of the screw with a temperature set between 20° C. and 70° C., then the kneading elements with a temperature ranging between 90° C. and 150° C. and finally the reverse pitch elements with temperatures ranging between 100° C. and 120° C.

This particular procedure generates a machine torque of 41% with an outlet pressure of 20 bars. The specific power of the system is approximately 17 KWh/kg.

The product is directed at the outlet to a die made up of 44×1.5 mm cylindrical holes, from which the textured protein is expelled, which is cut using knives rotating at 1,500 revolutions/minute placed flush with the outlet of the extrusion die.

The textured protein thus produced is dried in a 14×14 KM*1 VD dryer by Geelen Counterflow at a temperature of 88° C. in a 2,400 kg/h hot air flow.

A measurement of water holding capacity according to test A indicates a value of 3.8 g/g of water.

A density measurement of the extruded protein using test B indicates a value of 210 g/l.

### Example 2: Production of a Composition of Leguminous Proteins Textured in a Dry Process Outside of the Invention (Water to MS Ratio Too Low)

A powder mixture consisting of 87% of NUTRALYS® F85M pea protein (comprising 87.2% of proteins) by ROQUETTE and 12.5% of 150M pea fiber is produced.

This mixture is introduced by gravity into a COPERION ZSK 54 MV extruder from COPERION.

The mixture is introduced with a regulated flow rate of 300 kg/h. An amount of 55 kg/h of water is also introduced. The water to powder mass ratio is therefore (55/300)*100=18.3%.

The extrusion screw, made up of 85% feeding elements, 5% kneading elements and 10% reverse pitch elements, is rotated at a speed within 575 rpm and sends the mixture to a die. As indicated in the description, the feeding elements were placed at the very beginning of the screw with a temperature set between 20° C. and 70° C., then the kneading elements with a temperature ranging between 90° C. and 150° C. and finally the reverse pitch elements with temperatures ranging between 100° C. and 120° C.

This particular procedure generates a machine torque of 65% with an outlet pressure of 25 bars. The specific power of the system is approximately 14 KWh/kg.

The product is directed at the outlet toward a die made up of 44×1.5 mm cylindrical holes, from which the textured protein is expelled, which is then cut using knives rotating at 2,100 revolutions/minute.

The textured protein thus produced is dried in a 14×14 KM*1 VD dryer at a temperature of 86° C. in a 2,000 kg/h hot air flow.

A measurement of water holding capacity according to test A indicates a value of 3.4 g/g of water.

A density measurement of the extruded protein using test B indicates a value of 115 g/l.

An additional test was carried out with the same parameters but the screw speed was increased to 1,075 revolutions/min: the density was even lower, at 103 g/L.

### Example 2a: Production of a Composition of Leguminous Proteins Textured in a Dry Process Outside of the Invention (Water to MS Ratio Too High)

A powder mixture consisting of 87% of NUTRALYS® F85M pea protein (comprising 87.2% of proteins) by ROQUETTE and 12.5% of 150M pea fiber is produced.

This mixture is introduced by gravity into a COPERION ZSK 54 MV extruder from COPERION.

The mixture is introduced with a regulated flow rate of 300 kg/h. An amount of 130 kg/h of water is also introduced. The water to powder weight ratio is therefore (55/300)*100=43.3%.

The extrusion screw, made up of 85% feeding elements, 5% kneading elements and 10% reverse pitch elements, is rotated at a speed within 575 rpm and sends the mixture to a die. As indicated in the description, the feeding elements were placed at the very beginning of the screw with a temperature set between 20° C. and 70° C., then the kneading elements with a temperature ranging between 90° C. and 150° C. and finally the reverse pitch elements with temperatures ranging between 100° C. and 120° C.

This particular procedure generates a machine torque of 35% with an outlet pressure of 15 bars.

The product is directed at the outlet toward a die made up of 44×1.5 mm cylindrical holes, from which the textured protein is expelled, which is then cut using knives rotating at 2,100 revolutions/minute.

The textured protein thus produced is dried in a 14×14 KM*1 VD dryer at a temperature of 86° C. in a 2,000 kg/h hot air flow.

A measurement of water holding capacity according to test A indicates a value of 1.5 g/g of water.

A density measurement of the extruded protein using test B indicates a value of 301 g/l.

### Example 3: Production of a Composition of Leguminous Proteins Textured in a Dry Process Outside of the Invention (Fiber to Protein Ratio Too Low)

A powder mixture consisting of 99% of NUTRALYS® F85M pea protein (comprising 87.5% of proteins) by ROQUETTE and 1% of 150M pea fiber is produced. The protein content in 100 g of mixture is therefore 99*0.80=79.2 g.

This mixture is introduced by gravity into a COPERION ZSK 54 MV extruder from COPERION.

The mixture is introduced with a regulated flow rate of 300 kg/h. An amount of 78 kg/h of water is also introduced. The water to powder mass ratio is therefore (78/300)*100=26%.

The extrusion screw, made up of 85% feeding elements, 5% kneading elements and 10% reverse pitch elements, is rotated at a speed within 1,000 rpm and sends the mixture to a die. As indicated in the description, the feeding elements were placed at the very beginning of the screw with a temperature set between 20° C. and 70° C., then the kneading elements with a temperature ranging between 90° C. and 150° C. and finally the reverse pitch elements with temperatures ranging between 100° C. and 120° C.

This particular procedure generates a machine torque of 40% with an outlet pressure of 19 bars.

The product is directed at the outlet to a die made up of 44×1.5 mm cylindrical holes, from which the textured protein is expelled, which is cut using knives rotating at 1,500 revolutions/minute placed flush with the outlet of the extrusion die.

The textured protein thus produced is dried in a 14×14 KM*1 VD dryer by Geelen Counterflow at a temperature of 88° C. in a 2,400 kg/h hot air flow.

A measurement of water holding capacity according to test A indicates a value of 3.4 g/g of water.

A density measurement of the extruded protein using test B indicates a value of 105 g/l.

### Example 4: Production of a Composition of Leguminous Proteins Textured in a Dry Process (Example of a Lower Cutting Speed)

A powder mixture consisting of 87.5% of NUTRALYS® F85M pea protein (comprising 80% of proteins) by ROQUETTE and 12.5% of 150M pea fiber is produced. The protein content in 100 g of mixture is therefore 87.5*0.80=70 g.

This mixture is introduced by gravity into a COPERION ZSK 54 MV extruder from COPERION.

The mixture is introduced with a regulated flow rate of 300 kg/h. An amount of 78 kg/h of water is also introduced. The water to powder mass ratio is therefore (78/300)*100=26%.

The extrusion screw, made up of 85% feeding elements, 5% kneading elements and 10% reverse pitch elements, is rotated at a speed within 1,000 rpm and sends the mixture to a die. As indicated in the description, the feeding elements were placed at the very beginning of the screw with a temperature set between 20° C. and 70° C., then the kneading elements with a temperature ranging between 90° C. and 150° C. and finally the reverse pitch elements with temperatures ranging between 100° C. and 120° C.

This particular procedure generates a machine torque of 60% with an outlet pressure of 23 bars.

The product is directed at the outlet toward a die made up of 44×1.5 mm cylindrical holes, from which the textured protein is expelled, which is then cut using knives rotating at 500 revolutions/minute placed flush with the outlet of the extrusion die.

The textured protein thus produced is dried in a 14×14 KM*1 VD dryer by Geelen Counterflow at a temperature of 88° C. in a 2,400 kg/h hot air flow.

A measurement of water holding capacity according to test A indicates a value of 3.8 g/g of water.

A density measurement of the extruded protein using test B indicates a value of 209 g/l.

### Example 5: Comparison of the Compositions of Leguminous Proteins Textured in a Dry Process Obtained in the Above Examples and of Compositions Derived from the Prior Art

The protocols described above in the description are implemented in order to measure the density according to test B, the water holding capacity according to test A, as well as the size of the constituent particles measured according to test C.

The samples obtained in Examples 1 to 4 are compared, as is a selection of textured proteins on the market.

TABLE 1

| | | | Water holding | | | |
|---|---|---|---|---|---|---|
| | Moisture | Density | capacity | % size | % size | % size |
| Example | (by % weight) | (g/l) | (g/g) | 5 to 10 mm | 2 to 5 mm | 0 to 2 mm |
| Example 1 According | 10.1 | 210 | 3.8 | 0.4 | 90.7 | 8.8 |
| to the invention | | | | | | |
| Example 2 Lower | 7.5 | 115 | 3.4 | 8.8 | 79 | 13.3 |
| water to MS ratio | | | | | | |
| Example 2a Higher | 8.1 | 301 | 1.5 | undetermined | | |
| water to MS ratio | | | | | | |
| Example 3 Lower fibers | 8.5 | 105 | 3.4 | undetermined | | |
| to proteins ratio | | | | | | |
| Example 4 Lower | 8.6 | 209 | 3.8 | 24 | 73.6 | 2.2 |
| cutting speed | | | | | | |
| Nutralys T70S | 8.2 | 120 | 2.5 | 75 | 10 | 3 |
| (Roquette, pea) | | | | | | |
| Bona Vita (Sojovy | 10 | 300 | 3.4 | 17 | 73 | 9 |
| Granulat, soybean) | | | | | | |
| Trutex (MGP, soybean) | 9 | 260 | 2.5 | 25 | 61 | 14 |

Thus, it can be seen that only the product according to Example 1 allows a composition to be obtained with a Water Holding Capacity according to test A that is greater than 3.5 g of water per gram of dry proteins. The composition of Example 1 is unique because it has a high water holding capacity but with a density higher than 200 g/l. Furthermore, the particle size distribution is satisfactory in that at least 85% of particles are between 2 and 5 mm in size.

### Example 6: Use of a Composition of Leguminous Proteins Textured in a Dry Process According to the Invention in Meat Analogs

A hamburger or burger patty is produced using the compositions presented in the examples.

The ingredients used are as follows (the amounts indicated in the table below are given in grams per 100 g of finished burger):

TABLE 2

| Ingredients | Burger recipe #1 |
|---|---|
| Drinking water | 53.55 |
| Textured protein | 19.5 |
| Crushed ice | 6 |
| Methyl cellulose | 2 |
| Onions | 5.9 |
| Sunflower oil | 5.4 |
| Native potato | 2 |
| starch | |
| Pea Fiber I50 | 3 |
| (Roquette) | |
| Garlic powder | 0.5 |
| Salt | 0.2 |
| Black pepper | 0.1 |

The production procedure is as follows:

1. Hydrate the textured proteins in drinking water for 30 min.
2. Only for the burger with NUTRALYS T70S (outside of the invention—line 3 of table 1), mill the textured protein/water mixture for 45 seconds using a KENWOOD FDM302SS automatic mixer (speed 1), then leave in contact with water for a further 30 minutes.
3. Mix the methyl cellulose and the crushed ice in a container, then place in a refrigerator for 5 minutes.
4. Mix all the other ingredients in another container.
5. Combine the mixtures obtained in steps 1 (or 2), 3 and 4 in the same container, and mix in order to obtain a homogeneous composition.
6. Form the burger patties by hand with the final mixture with an amount of approximately 150 g.

After tasting by a panel of 10 people, it is acknowledged that the burger made with the textured protein according to the invention is closer to a burger made from animal meat than a burger made with NUTRALYS® T70S: the fibrous sensation is more present during tasting, less rubbery.

It is very surprising because of the prior knowledge (see paragraph 18 referring to the article entitled “Effect of soy particle size and color on the sensory properties of ground beef patties”) to obtain a better organoleptic result with the protein textured according to the invention, which has a smaller particle size than the NUTRALYS® T70S textured pea protein. It is the precise and specific selection of the water holding capacity and density features that allows this excellent result to be obtained with this small particle size and without the shredding step.

The panel mainly deems that the burger obtained with the textured protein according to Example 3 yields a softer, more rubbery result, and therefore that is not as close as with the protein according to the invention.

The panel also mainly deems that the burger obtained with the textured protein according to Example 4 provides an external appearance that is rather different than the control recipe, by showing larger particles.

### Example 7: Use of a Composition of Leguminous Proteins Textured in a Dry Process According to the Invention in a Bolognaise Sauce

A bolognaise sauce is produced using the compositions presented in the examples.

The ingredients used are as follows (the amounts indicated in table 3 below are provided in grams per 100 g of finished sauce):

TABLE 3

| Ingredients | Bolognaise sauce recipe |
|---|---|
| Drinking water | 56.10 |
| Textured protein | 5.5 |
| Apple extract | 0.16 |
| Tomato concentrate | 33.2 |
| Vinegar | 0.83 |
| Salt | 0.91 |
| CLEARAM ® CH3020 | 1.82 |
| starch (ROQUETTE) | |
| Provencal herbs | 0.3 |

The production procedure is as follows:

1. Mix all the ingredients in a HotmixPro Creative mixer.
2. Cook at 90° C. for 10 min on speed 2.
3. Fill a canning jar with the resulting sauce.
4. Sterilize for 1 hour at 120° C. using a Steriflow® sterilizer.

A comparative example was carried out. According to this comparative example, the textured protein according to the invention is replaced by NUTRALYS T70S in the above bolognaise sauce recipe.

After tasting by a panel of 10 people, it is acknowledged that the bolognaise sauce made with the textured protein according to the invention is closer to a bolognaise sauce made from animal meat than a bolognaise sauce made with NUTRALYS T70S: when tasting, the presence of large particles is not felt as much.

The panel mainly deems that the bolognaise sauce obtained with the textured protein according to Example 4 provides a result that is not as close as with the textured protein according to the invention because the feeling of large particles is greater.

### Example 8: Use of a Composition of Leguminous Proteins Textured in a Dry Process According to the Invention for Producing a Plant-Based Sausage

A plant-based sausage is produced using the compositions presented in the examples.

The ingredients used are as follows (the amounts indicated in table 4 below are provided in grams per 100 g of finished sausage):

TABLE 4

| Ingredients | Sausage recipe #1 |
|---|---|
| Drinking water | 50.02 |
| Textured protein | 17.47 |
| Egg white | 4.48 |
| I50M pea fiber | 0.91 |
| (Roguette) | |
| Native potato | 1.73 |
| starch (ROQUETTE) | |
| PREGEFLO P100 | 0.91 |
| potato starch | |
| (ROQUETTE) | |
| Wheat gluten | 1.73 |
| (ROQUETTE) | |
| Bread crumbs | 1.73 |
| NUTRALYS ® F85F | 0.91 |
| pea protein isolate | |
| (ROQUETTE) | |
| Sunflower oil | 7.79 |
| 10*10 red pepper cubes | 8.81 |
| Chopped onions | 0.96 |

The production procedure is as follows:

1. On the one hand, hydrate the textured protein composition according to the invention for 30 minutes in water.
2. On the other hand, mix all the powders together.
3. Add the above two mixtures to the bowl of a Kenwood, also along with the sunflower oil, peppers and onion.
5. Mix for 3 minutes on speed 1.
6. Introduce the mixture into artificial casings.
7. Cool in fresh water (10° C.) then peel off the artificial casings.

A comparative example was carried out. According to this comparative example, the textured protein according to the invention is replaced by NUTRALYS T70S in the above sausage recipe.

After tasting by a panel of 10 people, it is acknowledged that the sausage made with the textured protein according to the invention is closer to a sausage made from animal meat than a sausage made with NUTRALYS T70S: tasting the internal composition is much more homogeneous.

As with the previous example, it is very surprising because of the prior knowledge (see paragraph 18 referring to the article entitled “Effect of soy particle size and color on the sensory properties of ground beef patties”) to obtain a better organoleptic result with the textured protein according to the invention, which has a smaller particle size than the NUTRALYS® T70S textured pea protein. It is the precise and specific selection of the water holding capacity and density features that allows this excellent result to be obtained with this small particle size and without the shredding step.

### Example 9: Use of a Composition of Leguminous Proteins Textured in a Dry Process According to the Invention to Produce Crispy Muesli (or “Crunchy Clusters”

A crispy muesli is produced using the compositions presented in the examples.

The ingredients used are as follows (the amounts indicated in table 5 below are provided in grams per 100 g of finished sausage):

TABLE 5

| | | | Recipe with the |
|---|---|---|---|
| | | Recipe with the | textured pea |
| | | textured pea | proteins of |
| Ingredients | Control | proteins of | the prior art |
| (in g) | recipe | example 1 | (NUTRALYS ® T70S) |
| Rolled oats | 40 | 28 | 28 |
| (Quaker Oats) | | | |
| Puffed rice | 10 | | |
| (Rice Krispies, | | | |
| Kellogg's) | | | |
| Cornflakes | 10 | 10 | 10 |
| (Kellogg's | | | |
| Cornflakes) | | | |
| Textured pea | | 22 | 22 |
| protein | | | |
| Sucrose | 17 | | |
| Water | 10 | | |
| Sunflower oil | 8 | | |
| Glucose syrup | 5 | | |
| (Glucose syrup | | | |
| 6080, ROQUETTE) | | | |
| Total | 100 | 100 | 100 |

The production procedure is as follows:

1. Mix the sucrose, water, glucose syrup and oil to prepare a syrup by heating and stirring with a Hotmix mixer, speed 2 at 85° C. (the weight can be checked to avoid/correct any water evaporation).
2. Add the other ingredients and mix on speed 1 using a Kitchen Aid Artisan 5KSM175PS.
3. Spread out on a baking tray and bake at 140° C. for 25 minutes.

After tasting by a panel of 10 people, it is acknowledged that the crispy muesli made with the textured protein according to the invention is closer to the crispy muesli control recipes than a crispy muesli made with NUTRALYS T705. Indeed, the various ingredients of the cluster are deemed to be more loosely related with NUTRALYS® T705.

The panel mainly deems that the crispy mueslis obtained with the textured protein according to Example 4 are also deemed to be more loosely related.

1. A composition comprising leguminous proteins textured in a dry process in the form of particles, the composition having a water holding capacity measured by a test A of more than 3.5 g of water per g of dry proteins, preferably ranging between 3.5 and 4.5 g of water per g of dry proteins, even more preferably ranging between 3.5 and 4 g of water per g of dry proteins, a density measured by a test B ranging between 190 and 230 g/l and at least 85% of the textured leguminous protein particles being between 2 mm and 5 mm in size.

2. The composition of leguminous proteins textured in a dry process according to claim 1, wherein the leguminous protein is selected from the list consisting of faba bean protein and pea protein.

3. The composition of leguminous proteins textured in a dry process according to claim 1, wherein the protein content of the composition ranges between 60% and 80%, preferably between 70% and 80% by dry weight.

4. The composition of leguminous proteins textured in a dry process according to claim 1, wherein it has a dry matter content of more than 80% by weight, preferably of more than 90% by weight.

5. A method for producing a composition comprising leguminous proteins according to claim 1, the method comprises the following steps:
1) providing a powder comprising leguminous proteins and leguminous fibers, having a dry weight ratio of leguminous proteins to leguminous fibers ranging between 70/30 and 90/10, preferably ranging between 75/25 and 85/15;
2) extrusion cooking the powder with water, the water to powder mass ratio before cooking ranging between 20% and 40%, preferably between 25% and 35%, even more preferably 30%;
3) cutting the extruded composition at the extruder outlet made up of an outlet die with holes, with a diameter of 1.5 mm and equipped with a knife, the speed of rotation of which ranges between 1,200 and 1,800 revolutions per minute, or between 2,000 and 2,400 revolutions per minute, preferably around 1,500 revolutions per minute; and
4) drying the composition thus obtained.

6. The production method according to claim 5, wherein the leguminous protein is a pea protein.

7. The production method according to claim 6, wherein the pea protein has a protein content advantageously ranging between 60% and 90%, preferably between 70% and 85%, even more preferably between 75% and 85% by weight of the total dry matter.

8. The production method according to claim 6, wherein the pea protein is characterized by a particle size characterized by a Dmode ranging between 150 microns and 400 microns, preferably between 150 microns and 200 microns or between 350 microns and 450 microns.

9. The production method according to claim 5, wherein the leguminous fiber contains between 40% and 60% of polymers made up of cellulose, hemicellulose and pectin, preferably between 45% and 55%, as well as between 25% and 45% of pea starch, preferably between 30% and 40%.

10. The production method according to claim 5, wherein step 2 is carried out by extrusion cooking in a twin-screw extruder characterized by a length to diameter ratio ranging between 35 and 45, preferably 40, and equipped with a series of 85-95% feeding elements, 2.5-10% kneading elements, and 2.5-10% reverse pitch elements.

11. The production method according to claim 10, wherein the feeding elements will be placed at the very beginning of the screw with a temperature set between 20° C. and 70° C., then the kneading elements with a temperature ranging between 90° C. and 150° C. and finally the reverse pitch elements with temperatures ranging between 100° C. and 120° C.

12. The production method according to claim 10, wherein the screw is rotated between 900 and 1,200 revolutions per minute, preferably between 1,000 and 1,100 revolutions per minute.

13. The production method according to claim 5, wherein a specific power ranging between 10 and 25 kWh/kg is applied to the powder mixture, by regulating the pressure at the outlet in a range ranging between 10 and 25 bars, preferably between 12 and 16 bars.

14. A use of a composition of leguminous proteins textured in a dry process according to claim 1, in an industrial application selected from the human and animal food industry, industrial pharmaceuticals or cosmetics.

15. The use according to claim 14, wherein the leguminous protein is a pea protein.
